(19) United States
(12) Patent Application Publication
(10) Pub. No.: US 2026/0300753 A1
(43) Pub. Date: Oct. 1, 2026

(54) FEDERATED LEARNING WITH HARD EXAMPLES

(71) Applicant: KONINKLIJKE PHILIPS N.V., EINDHOVEN (NL)

(72) Inventors: ALEKSANDR BUKHAREV, EINDHOVEN (NL); ANCA IOANA DANIELA BUCUR, WAALRE (NL); HANS-ALOYS WISCHMANN, EINDHOVEN (NL); RICHARD VDOVJAK, WAALRE (NL); IRINA FEDULOVA, EINDHOVEN (NL); SHIVA MOORTHY POOKALA VITTAL, BANGALORE (IN); ANSHUL JAIN, BANGALORE (IN); SHREYA ANAND, BANGALORE (IN); NIKOLAY PROKOPTSEV, EINDHOVEN (NL); RACHAKONDA SIDDARTHA, BANGALORE (IN)

(21) Appl. No.: 18/881,331

(22) PCT Filed: Jun. 9, 2023
(86) PCT No.: PCT/EP2023/065455

§ 371 Date: Jan. 6, 2025

(30) Foreign Application Priority Data
Jul. 6, 2022 (RU) 2022118388

(51) Int. Cl. G06N 3/098; G16H 10/60; G16H 30/00

(52) U.S. Cl. CPC G06N 3/098; G16H 10/60; G16H 30/00

(57) ABSTRACT

The invention relates to a federated learning system (300) for federated learning of a machine learnable model (MLM). A local system (LS) obtains a hard example (HEDi) for the machine learnable model (MLM), and determines a model update for updating model parameters of the machine learnable model from the hard example (MU). The local system (LS) provides, to the coordinator system (CS), a hard example descriptor comprising the model update (MU) and metadata (HEDi) describing the hard example. The coordinator system collects hard example descriptors (HEDi) and clusters them. Based on the model updates (MU) and the metadata comprised in the hard example descriptors of the cluster (HEDi), the coordinator system (CS) accepts the cluster for use in the federated learning, and provides an acceptance notice (AN) to the local system (LS) which then uses the hard example in the federated learning of the machine learnable model (MLM).

### FIELD OF THE INVENTION

The invention relates to a federated learning system, to a local system and a coordinator system for use in the federated learning system, and to computer-implemented methods of operating the local system and the coordinator system, respectively. The invention further relates to a computer-readable medium.

### BACKGROUND OF THE INVENTION

The performance of Machine Learning (ML) models in general and Deep Learning (DL) models in particular depends on the quality and the content of the data used for training and validation. It has been reported in the literature that DL models can overfit on subtle institutional data biases and can show low performance on data from institutions whose data were not seen during training, see, e.g., M.J. Sheller et al., “Federated learning in medicine: facilitating multi-institutional collaborations without sharing patient data”, Nature Sci Rep 10, 12598 (2020). Even when the training and testing datasets are large, diverse, representative, and perfectly annotated, an ML/DL algorithm may still fail after deployment once it faces a rare (in the training dataset) or previously unseen case.

The concept of post-product learning, also known as active learning or lifelong learning (LL) aims to address this problem. According to the Lifelong Learning (LL) concept, rare or complex (and potentially valuable) examples may be identified, mined and added to the reference (training and validation) datasets for future re-training. In particular, in the medical setting, where models are applied on healthcare data about patients, the need for post-product learning has been recognized by the United States Food and Drug administration in the discussion paper “Proposed Regulatory Framework for Modifications to Artificial Intelligence/Machine Learning-Based Software as a Medical Device”, which discusses adaptive Al solutions in healthcare that can learn from real-world data and post-deployment performance monitoring. When applying post-product learning, especially in the healthcare setting, privacy, custodianship and data governance are important concerns.

Although healthcare providers may be interested to contribute to and then use an improved model, they are in most cases not willing or legally not allowed to share the clinical data with each other nor with the model developer/vendor.

A known technique that can be used to alleviate some privacy, security and administrative concerns in training a model using data from different parties is federated learning. In federated learning, a machine learnable model, such as a deep neural network, can be updated with new local data samples from respective decentralized systems, without exchanging them. This is sometimes referred to as the “model-to-data” paradigm. For example, in the healthcare setting, the machine learnable model may be deployed at respective healthcare providers (e.g., locally, or in multiple segregated cloud instances), where it may be used to make inferences about patient data for the provision of care.

Federated learning allows the machine learnable model to be updated based on patient data of these respective healthcare providers without the need to move this data out of the institution's administrative domain.

### SUMMARY OF THE INVENTION

A problem that occurs when updating a machine learnable model with additional examples after deployment, is that it is not clear whether using the additional examples will improve the model. The training and validation datasets are typically comprised of diligently compiled, annotated, and curated cases. On the other hand, real-world cases, for example coming from regular clinical care, may be more prone to errors, for example, due to a lack of final case outcomes for ground truths, an intense work environment with a high workload and time pressure, and lower levels of training and experience of the healthcare providers, compared to an academic study environment.

Moreover, there are significant differences across institutions with respect to data collection, protocols, techniques and collected ground truth which may cause problems when all such data is collected in one large dataset.

It would thus be desirable to provide federated learning techniques that provide additional assurance that additional training examples improve the performance of the model being trained.

In accordance with a first aspect of the invention, a federated learning system is provided, as defined by claim 1. In accordance with further aspects of the invention, a local system and a coordinator system for use in the federated learning system are provided, as defined by claims 11 and 12, respectively. In accordance with still further aspects of the invention, computer-implemented methods of operating a local system and a coordinator system are provided, as defined by claims 13 and 14, respectively. In accordance with an aspect of the invention, a computer-readable medium is provided as defined by claim 15.

Various aspects relate to the use of federated learning to train a machine learnable model, in particular, to update a trained machine learnable model based on additional training examples. Federated learning refers to the training of a machine learnable model using a distributed protocol without the exchange of training examples. Using federated learning, the machine learnable model may be updated based on additional training examples from multiple respective systems, without these systems needing to explicitly exchange these additional training examples with others. These systems are referred to herein as “local systems”. Such local systems are also sometimes referred to as “local deployments”, “local nodes”, “nodes”, or “edge devices”, depending on the concrete setting. Generally, federated learning can operate both across silos and across devices. The respective local systems are typically operated by respective organizations.

To update the model, the local systems may determine respective model updates, e.g., updated model parameters, to the machine learnable model, based on their local training examples, and may exchange these within the federated learning system. The updated model may be derived from these model updates. The federated learning system can for example be a centralized federated learning system, in which a central server coordinates the federated learning, or a decentralized federated learning, in which there is no such central server.

In various aspects, a local system may obtain a hard example for the machine learnable model. Generally, a hard example is a model input that has been identified at that local system as a candidate input for use in the federated learning of the machine learnable model and/or for use in a reference dataset for the machine learnable model as also described elsewhere. For example, the model input may be identified as a hard example because the model input challenging for the model, e.g., the model provides an incorrect result for the model input, and/or the model provides a low-confidence output for the model input.

In some cases, using the hard example may lead to an improved machine learnable model (e.g., in terms of robustness and/or generalizability) and/or reference dataset, whereas in other cases, it may lead to a deterioration of the utility of the model. For example, the hard example may represent an interesting and rare case, also referred to herein as a fascinoma. Using a fascinoma is generally beneficial to improve the coverage of the model and/or reference dataset. On the other hand, the hard example may represent ambiguous data, e.g., may contain artifacts and/or noise, and/or may be incorrectly annotated. In such cases, using the hard example in the federated learning and/or the reference dataset may lead to performance degradations.

Accordingly, the inventors envisaged to use a coordinator system in the federated learning system that decide whether to accept a given hard example, e.g., to use the corresponding local updates or to reject it. This way, the risk of updates that would pollute the machine learnable model, is minimized. The coordinator system can be the central system of a centralized federated learning system, for example, but can also be one of the local systems or another system.

The coordinator system may decide which hard examples from the local systems to accept or reject, but interestingly it may make this decision not based on the model inputs of the hard examples themselves (since it may be undesirable for the coordinator system to inspect them), but based on hard example descriptors. A hard example descriptor may comprise a model update for updating the model parameters of the machine learnable model from the hard example, as well as metadata describing the hard example, but not the model input itself. Accordingly, the hard example descriptor may be provided by the local system to the coordinator system without the privacy concerns that would arise if the model input itself would have been shared. Still, a relatively good assessment of whether the example should be used or not, may be determined.

In particular, the model update may be used to estimate an effect of the example on the accuracy of the machine learnable model according to a current reference dataset, whereas the metadata may be used to determine whether the example represents a rare case underrepresented in the data that was used to train the model and/or in the current reference dataset. Model updates may be accepted according to their having a positive effect and/or being underrepresented. In particular, the inventors realized that metadata may allow to differentiate rare cases from low quality and in particular mislabeled data: rare cases generally have rare metadata as well, whereas low quality data may have similar metadata to similar but higher quality records.

Accordingly, it may be assured that the performance of the model is improved by training with large and diverse datasets, including the addition of rare cases, without being affected by performance drift due to for instance low quality labels or data used during re-training. In other words, using the provided techniques, fascinoma may be determined in accordance with meta-data and historical recordings, and relevant rare cases versus erroneous cases may be identified based on similar occurrences across different sites using a similarity evaluation of hard cases with respect to impact on model and source of deviation from the standard cases.

Specifically, in order to decide which hard examples to accept or reject, the inventors envisaged for the coordinator system to select a cluster of similar hard example descriptors from the set of hard example descriptors that it has collected from the local systems of the federated learning system. The coordinator system may then decide to accept or reject the cluster based on the metadata and the model updates of the hard examples in the cluster.

The use of a cluster of similar hard example descriptors has a number of advantages. One advantage is that the clustering itself provides valuable information to decide whether or not to accept the hard examples in the cluster. For example, the size of the cluster and/or the number of local systems contributing to the cluster are informative of whether accepting the cluster is likely to have a positive impact, e.g., hard examples that represent isolated measuring errors or misclassifications are less likely to occur in a cluster of sufficient size and/or that spans sufficient different local systems. Also other features that are indicative of whether hard examples are beneficial are not, such as an estimated effect on accuracy can be more reliably estimated for a cluster than for individual hard examples.

Having accepted a number of hard examples for use in the federated learning, the coordinator system may notify the respective local systems from which the hard example descriptors were collected. The machine learnable model may be updated by these local systems by performing a federated learning update of the model using the accepted hard examples from one or more clusters. For this, techniques that are known per se from federated learning may be used. Interestingly, these techniques are applied in a different way than in traditional federated learning, for example in that the training may be performed stretched out in time; in that the training may be initiated not by a coordinator choosing a next local system for training, but by a local system obtaining one or more hard examples and providing their descriptors to the coordinator; and in that the hard examples may not be immediately applied to the machine learnable model but instead processed by a decision procedure that accepts or rejects them, directly or at a later point in time.

In some cases, all hard examples of a cluster may be used in the federated learning. In other cases, only a subset of the hard examples may be used, e.g., to balance the training dataset and/or to use further hard examples of the cluster in a reference dataset as described herein.

Optionally, the machine learnable model may be applied to a model input comprising healthcare data about a patient. In particular, the machine learnable model may be an image classifier, e.g., for radiology images; or another type of image processing model. In another example, the machine learnable model may be used to classify electronic medical records (EMRs). In such examples, the respective local systems may be operated by healthcare providers such as hospitals. The coordinator system may be operated by a third party. Interestingly, according to the provided techniques, the coordinator system may not need to see the hard examples to decide whether to accept or reject them and it may thus be feasible to use the provided techniques on medical data despite its sensitivity.

In the healthcare sector, there is an increased interest, including from regulatory authorities such as the FDA, to adopt solutions that include Al models to improve performance and increase automation. Avoiding performance drift of the model post-deployment is an important issue in this setting. Moreover, it may be required in the healthcare sector to prove that the performance of Al products is similarly high in diverse production environments as in the validation phase (post-marketing) and it does not deteriorate over time. This makes the techniques particularly useful in the medical domain.

Optionally, the coordinator system may accept a cluster based on one or more of a size of the cluster, a number of local systems contributing to the cluster, an estimated effect of the cluster on an accuracy of the machine learnable model and a degree to which the cluster is representative of the reference dataset. These aspects may all be indicative of whether it is beneficial to use the cluster. Preferably, at least the size or number of systems, the accuracy estimate, and the representativeness degree are used. A larger cluster or a larger number of systems contributing may make the cluster more relevant and less likely to be an artifact. The effect on accuracy may indicate generally, also for non-rare cases, whether training on them is beneficial. The degree of representativeness may indicate a likelihood of the cluster representing a rare case. In particular, the cluster may be accepted based on the cluster being sufficiently unrepresentative of the reference dataset, in other words having a representativeness degree not exceeding a threshold, for example, in combination with the cluster being of sufficient quality as indicated by the cluster size and/or number of systems, and/or an additional check whether the metadata is not anomalous.

Optionally, one or more hard examples of an accepted cluster may be used for the federated learning, and one or more further hard examples may be included in a reference dataset of the coordinator system. Interestingly, because both types come from the same cluster, they may be similar to each other, and so both a better-trained model and a reference dataset more suitable for evaluating this better-trained model can be obtained. The coordinator system may provide an acceptance notice of a hard example to the respective local system indicating the intended use: for training or inclusion in the reference dataset. Depending on this indication, the local system may use the hard example in the federated learning or provide the hard example to the coordinator system. Although in this case privacy-sensitive data is shared with the coordinator system, the amount of records that need to be shared is still greatly introduced, and it may also be easier to get permission to share the example in the specific case that it is found to be a hard example that is needed for the reference dataset.

Optionally, the local system may be configured to obtain a model input for the machine learnable model; compute a confidence score for the model input; and select the model input as the hard example based on the confidence score is below a threshold, e.g., the input may be selected if the confidence score is below the threshold, in combination with one or more other conditions. Low confidence may indicate that the model input is potentially useful to train or validate the model, e.g., low confidence may be expected for rare cases underrepresented in the training dataset. For example, the confidence score may be determined by the machine learnable model itself or by a separate model. Computing the confidence score may be combined with applying the model to the model input, for example, during regular use of the model. If the model input is selected as hard example, the hard example may then be manually checked or annotated, for example.

Instead of in addition to using low-confidence model inputs, one or more hard examples may also be obtained for example by a user flagging an output of the machine learnable model as incorrect, and/or by randomly selection among model inputs to which the model is applied and/or be manual selection by a user.

Optionally, the metadata may comprise data that is derived from neither the model input nor the label, e.g., the ground-truth model output, of the hard example. For example, the model input may be an image and the metadata may comprise metadata of the image, e.g., information about a scan protocol, equipment used, etc. Such metadata may be combined with metadata that is based on the model input and/or label. For example, the metadata may comprise one or more of a timestamp, a descriptor of the local system, a descriptor of a labeler of the hard example, a degree of agreement about a labelling of the hard example, information about a health care professional associated with the hard example, information about a patient associated to the hard example, and information about image acquisition settings the hard example.

Optionally, the coordinator system is configured to obtain feedback on the accepted cluster and to update criteria for accepting clusters based on the feedback. For example, decisions about whether a cluster was accepted or rejected may be manually reviewed and/or may be validated with data that became available later. In such cases, criteria, in other words, parameters, that were used for the acceptance may be updated. This can comprise adapting a threshold, for example, but can also involve retraining a trainable model used to make the accept/reject decision.

Optionally, the coordinator system may be configured to keep a hard example descriptor that is not comprised in a selected cluster, or that is comprised in a cluster that was not accepted. The kept hard example descriptor may be used in a further cluster selection and acceptance. For example, a hard example that initially seems like an anomaly may later turn out to be a valuable hard example, if more cases similar to it arise later. For example, a cluster containing the hard example may initially be too small to be accepted but may later reach a sufficient size and/or number of parties contributing to it. In such cases, keeping the hard example can allow it to be used later in such a larger cluster and thus potentially be accepted and used as described herein. As another example, the metadata of the hard example descriptor may be updated based on newly available information, and the hard example descriptor may be accepted based on the updated metadata.

Optionally, following the federated learning based on an accepted hard example, the local system and/or other local systems may be configured to apply the machine learnable model updated based on the accepted hard example to a model input. Thus, an output of the machine learnable model can be obtained that benefits from the improved training using the hard example.

It will be appreciated by those skilled in the art that two or more of the above-mentioned embodiments, implementations, and/or optional aspects of the invention may be combined in any way deemed useful.

Modifications and variations of any system and/or any computer readable medium, which correspond to the described modifications and variations of a corresponding computer-implemented method, can be carried out by a person skilled in the art on the basis of the present description.

### BRIEF DESCRIPTION OF THE DRAWINGS

These and other aspects of the invention will be apparent from and elucidated further with reference to the embodiments described by way of example in the following description and with reference to the accompanying drawings, in which:

FIG. 1 shows a coordinator system for use in federated learning of a model;

FIG. 2 shows a local system for use in federated learning of a model;

FIG. 3 shows a federated learning system;

FIG. 4 shows a detailed example of how to train a machine learnable model by federated learning;

FIG. 5 shows a detailed example of how to accept a set of hard example descriptors for use in federated learning and/or for inclusion in a reference dataset;

FIG. 6 shows a computer-implemented method of operating a local system;

FIG. 7 shows a computer-implemented method of operating a coordinator system;

FIG. 8 shows a computer-readable medium comprising data.

It should be noted that the figures are purely diagrammatic and not drawn to scale. In the figures, elements which correspond to elements already described may have the same reference numerals.

### DETAILED DESCRIPTION OF EMBODIMENTS

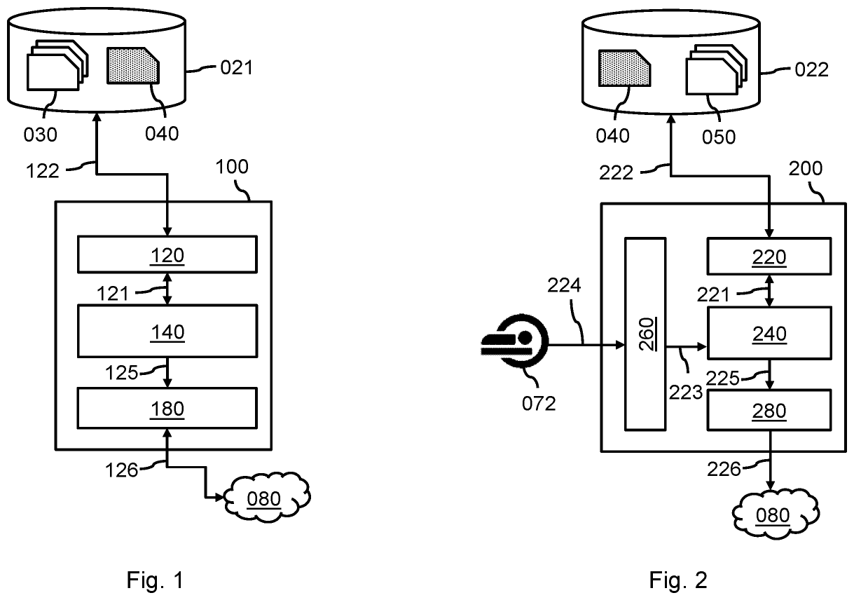

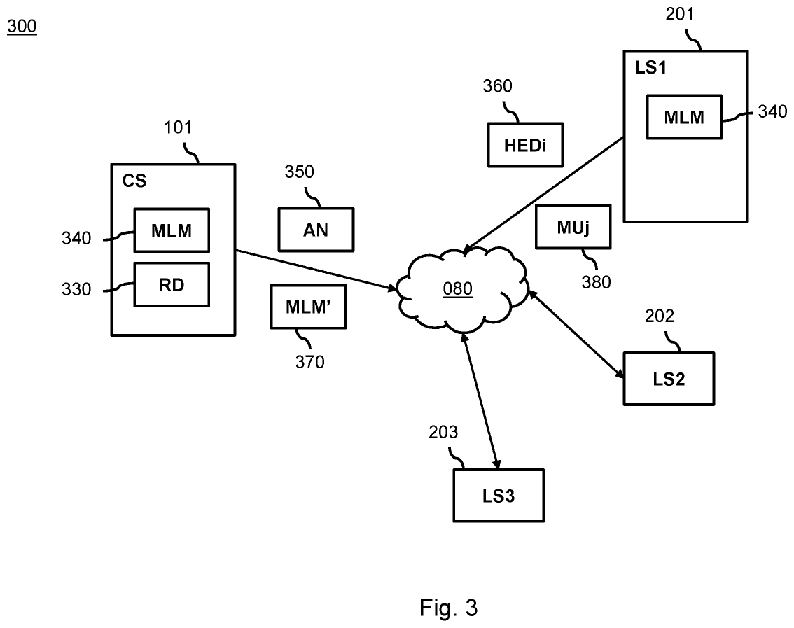

FIG. 1 shows a coordinator system 100 for use in a federated learning system. The federated learning system may be for federated learning of a machine learnable model. The federated learning system may comprise multiple local systems. For example, the federated learning system may be as described for FIG. 3. The coordinator system 100 may be combined with a local system.

The system 100 may comprise a data interface 120 for accessing a set 030 of hard example descriptors. A hard example descriptor may comprise a model update and metadata about a hard example. The system 100 may collect hard examples from the local systems of the federated learning system. For example, the system 100 may be capable to collect at least 100, at least 1000, or at least 10000 hard example descriptors. The system 100 may be able to collect the hard example descriptors from multiple local systems, e.g., the set 030 may contain hard example descriptors from multiple local systems.

The data interface 120 may be further for accessing model data 040 representing model parameters of the machine learnable model. As also described elsewhere, the coordinator system 100 may participate in the learning of the machine learnable model, for example, as a central system of a centralized federated learning system, and may accordingly be configured to pool model updates of respective local parties to obtain an updated version of the machine learnable model and to distributed the updated model to the local parties.

Generally, the machine learnable model may be any machine learnable model for which federated learning techniques are available. For example, the model may be a neural network. Neural networks are also known as artificial neural networks. Examples include deep neural networks and convolutional neural networks. In this case, the set of parameters 040 may comprise weights of nodes of the neural network. For example, the number of layers of the model may be at least 5 or at least 10, and the number of nodes and/or weights may be at least 1000 or at least 10000. Depending on the particular application, various known architectures for neural networks and other types of machine learnable models may be used. As a specific example, the machine learnable model may be an image classifier, e.g., for use in classifying medical images.

The data interface 120 may be further for accessing a reference dataset (not shown). The reference dataset is a set of, typically labelled, model inputs to the machine learnable model. The reference dataset may be used by the coordinator system 100 for processing hard example descriptors, and hard examples corresponding to the hard example descriptors may be added to the reference dataset, as described herein. The reference dataset may be used for additional purposes, e.g., some or all inputs of the reference dataset may be used as a validation dataset for testing the machine learnable model, e.g., in the federated learning, and/or some or all inputs of the reference dataset may be used as training data for training the machine learnable model by the coordinator system 100. The reference dataset may comprise at least 500, at least 1000, or at least 10000 inputs, for example. The reference dataset is preferably a high-quality dataset. For example, the reference dataset may be manually annotated, e.g., contains only labels that have been provided or at least checked by a human user.

As also illustrated in FIG. 1, the data interface 120 may be constituted by a data storage interface which may access the data, e.g., data 030 and/or 040, from a data storage 021. For example, the data storage interface 120 may be a memory interface or a persistent storage interface, e.g., a hard disk or an SSD interface, but also a personal, local or wide area network interface such as a Bluetooth, Zigbee or Wi-Fi interface or an ethernet or fiberoptic interface. The data storage 021 may be an internal data storage of the system 100, such as a hard drive or SSD, but also an external data storage, e.g., a network-accessible data storage. In some embodiments, the data 030, 040 may each be accessed from a different data storage, e.g., via a different subsystem of the data storage interface 120. Each subsystem may be of a type as is described above for data storage interface 120.

The system 100 may further comprise a processor subsystem 140 which may be configured to, during operation of the system 100, collect the set of hard example descriptors 030 from the multiple local systems. The processor subsystem 140 may be further configured to select a cluster of similar hard example descriptors from the set of hard example descriptors 030. The processor subsystem 140 may be further configured to, based on model updates and metadata comprised in the hard example descriptors of the cluster, accept the cluster for use in the federated learning. The processor subsystem 140 may be further configured to provide an acceptance notice for a hard example descriptor of the cluster to the local system from which the hard example descriptor was collected.

The system 100 may also comprise a communication interface 180 configured for communication 162 with the local systems of the federated learning system. Communication interface 180 may internally communicate with processor subsystem 140 via data communication 126. Communication interface 180 may be arranged for direct communication with the local systems, e.g., using USB, IEEE 1394, or similar interfaces. As shown, communication interface 180 may also communicate over a computer network 080, for example, a wireless personal area network, an internet, an intranet, a LAN, a WLAN, etc. For instance, communication interface 180 may comprise a connector, e.g., a wireless connector, an Ethernet connector, a Wi-Fi, 4G or 4G antenna, a ZigBee chip, etc., as appropriate for the computer network. Communication interface 180 may also be an internal communication interface, e.g., a bus, an API, a storage interface, etc.

The system 100 may further comprise an output interface for outputting trained data representing the learned model. For example, as also illustrated in FIG. 1, the output interface may be constituted by the data interface 120, with said interface being in these embodiments an input/output (‘IO’) interface, via which the trained model data may be stored in the data storage 021. For example, the model data defining the ‘untrained’ model may during or after the training be replaced, at least in part, by the model data of the trained model, in that the parameters of the dynamics model, such as weights and other types of parameters of neural networks, may be adapted to reflect the federated learning of the model. In other embodiments, the trained model data may be stored separately from the model data defining the ‘untrained’ model. In some embodiments, the output interface may be separate from the data storage interface 120, but may in general be of a type as described above for the data storage interface 120.

FIG. 2 shows a local system 200 for use in a federated learning system. The federated learning system may be for federated learning of a machine learnable model. The federated learning system may further comprise a coordinator system that is different from, or may be combined with, local system 200. For example, the federated learning system may be as described for FIG. 3.

Local system 200 may comprise a data interface 220 for accessing model data 040 representing model parameters of the machine learnable model. The model data may by as described for model data 040 of FIG. 1.

The system 200 may further comprise a processor subsystem 240. The processor subsystem 240 may be configured to apply the machine learnable model to one or more model inputs 050, for example, to output the result to a user or use the result to control a computer-controlled system.

Processor subsystem 240 may be further configured to, during operation of the system 200, obtain a hard example for the machine learnable model. For example, one of the model inputs 050 to which the machine learnable model is applied, may be determined to be a hard example. The obtaining of the hard example may comprise obtaining a human-determined or at least human-verified labelling of the hard example, e.g., it may be automatically determined that a model input is a hard example following which a human may provide the verified labelling, or it may be determined that a model input is a hard example based on a human labelling not matching the output of the model.

The processor subsystem 240 may be further configured to determine a model update for updating the model parameters of the machine learnable model from the hard example. This typically uses the human-verified labelling. The processor subsystem 240 may be further configured to provide, to the coordinator system, a hard example descriptor comprising the model update and metadata describing the hard example. The processor subsystem 240 may be further configured to, upon obtaining, from the coordinator system, an acceptance notice for the hard example descriptor, use the hard example in the federated learning of the machine learnable model.

After the machine learnable model has been updated using the hard example, processor subsystem 240 may be configured to continue applying the, now updated, machine learnable model to one or more further model inputs 050, thus benefiting from the improved training of the model.

The system 100 may also comprise a communication interface 280 configured for communication 226 with the coordinator system and/or other local systems of the federated learning system, for example via a computer network 080. It will be appreciated that the same considerations and implementation options apply for the data interface 220, processor subsystem 240, and communication interface 280 as for the corresponding elements of FIG. 1. It will be further appreciated that the same considerations and implementation options may in general apply to the system 200 as for the system 100 of FIG. 1, unless otherwise noted.

As illustrated in the figure, the system 200 may optionally comprise a sensor interface 260 for obtaining sensor data 224 acquired by a sensor 072. For example, the sensor data 224 may be a medical image 224 captured by a medical imaging device 072, e.g., a CT scanner or an MRI scanner. The system 200 may be configured to apply the machine learnable model to a model input that comprises or is otherwise based on the sensor data 224. The sensor 072 may but does not need to be part of the system 200. The sensor 072 may have any suitable form, such as an image sensor, a lidar sensor, a radar sensor, a pressure sensor, a contain temperature sensor, etc. The sensor data interface 260 may have any suitable form corresponding in type to the type of sensor, including but not limited to a low-level communication interface, e.g., based on I2C or SPI data communication, or a data storage interface of a type as described above for the data interface 220.

In some embodiments, the system 200 may comprise an actuator interface 280 for providing control data to an actuator (not shown). Such control data may be generated by the processor subsystem 240 to control the actuator based on a model output of the machine learnable model. The actuator may be part of system 200. For example, the actuator may be an electric, hydraulic, pneumatic, thermal, magnetic and/or mechanical actuator. Specific yet non-limiting examples include electrical motors, electroactive polymers, hydraulic cylinders, piezoelectric actuators, pneumatic actuators, servomechanisms, solenoids, stepper motors, etc. In other embodiments, the system 200 may comprise an output interface to a rendering device, such as a display, a light source, a loudspeaker, a vibration motor, etc., which may be used to generate a sensory perceptible output signal based on a model output of the machine learnable model.

In general, each system described in this specification, including but not limited to the system 100 of FIG. 1 and the system 200 of FIG. 2, may be embodied as, or in, a single device or apparatus, such as a workstation or a server. The device may be an embedded device. The device or apparatus may comprise one or more microprocessors which execute appropriate software. For example, the processor subsystem of the respective system may be embodied by a single Central Processing Unit (CPU), but also by a combination or system of such CPUs and/or other types of processing units. The software may have been downloaded and/or stored in a corresponding memory, e.g., a volatile memory such as RAM or a non-volatile memory such as Flash. Alternatively, the processor subsystem of the respective system may be implemented in the device or apparatus in the form of programmable logic, e.g., as a Field-Programmable Gate Array (FPGA). In general, each functional unit of the respective system may be implemented in the form of a circuit. The respective system may also be implemented in a distributed manner, e.g., involving different devices or apparatuses, such as distributed local or cloud-based servers. In some embodiments, the system 200 may be part of a medical imaging system, and may be used to improve machine learnable outputs output or used by the medical imaging system.

FIG. 3 shows a federated learning system 300. The federated learning system may be for federated learning of a machine learnable model MLM, 340. The machine learnable model may be as described for FIG. 1 or elsewhere.

The federated learning system 300 may comprise multiple local systems. For illustration purposes, three local systems LS1, 201; LS2, 202; and LS3, 203 are shown. The number of local systems may be at most or at least 10, at most or at least 20, or at most or at least 50, for example. A local system LS1, LS2, LS3 may be as described for local system 200 of FIG. 2.

The respective local systems LSi may be operated by respective organizations where the machine learnable model MLM is deployed, for example, at respective healthcare organizations, where the machine learnable model MLM may be applied to healthcare data, in other words clinical data, about a patient, such as a medical image. The model may be deployed at additional sites that do not act as local systems. The model MLM may for example be deployed by an organization on a cloud or on the local site of the organization.

A local system LSi may be configured to apply the machine learnable model MLM in addition to acting as a local system of the federated learning system, e.g., to apply an image classifier MLM to a medical image to obtain a classification of the medical image. Instead or in addition to the local system LSi applying the machine learnable model, the model may be applied by other systems of the organization operating the local system LSi.

The model MLM may be used to make inferences from patient data, e.g., processing it for the provision of care, so that it remains privacy-sensitive data even when sent to a cloud (typically encrypted). The model may be deployed at multiple sites and/or on multiple, segregated cloud instances where it may be used on respective data during care delivery in a clinical context, e.g., fully identified. While healthcare organizations may be interested to contribute to and then use an improved model, they may in many cases not be willing or legally not allowed to share clinical data with each other nor with the model developer/vendor. In particular it might be impossible or not allowed to relocate large amounts of examples from local systems LSi to a central storage for models retraining.

In order to enable recurrent enhancement of the machine learnable model MLM in post-deployment scenarios, without the need to provide all training examples to a centralized third party, a federalized learning system 300 may be used. The federated learning system 300 may comprise a coordinator system CS, 101, e.g., as described for coordinator system 100 of FIG. 1. The coordinator system CS may be a central system of the federated learning system (also known as central server), but this is not needed. The coordinator system CS may be one of the local systems of the federated learning system.

The coordinator system CS and the local systems LSi may communicate over a computer network 080, e.g., a local network and/or the internet, using their respective communication interfaces, as also discussed for FIG. 1.

A local system LS1 may be configured to obtain a, typically labelled, hard example for the machine learnable model; to determine a model update for updating model parameters MLM, 340 of the machine learnable model from the hard example; and to provide, to the coordinator system CS, a hard example descriptor HEDi, 360, comprising the model update and metadata describing the hard example.

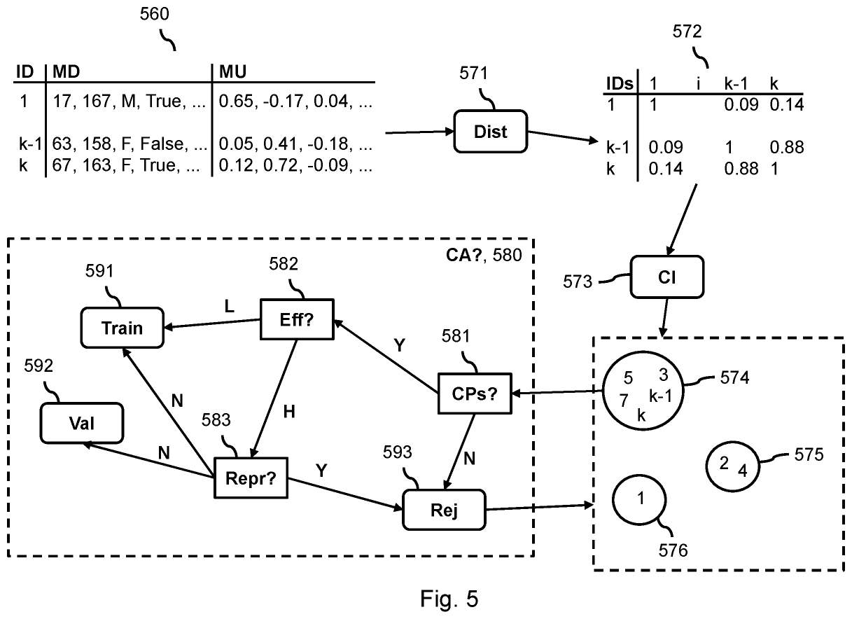

The coordinator system CS may be configured to collect a set of hard example descriptors HEDi from the multiple local systems LSi, and accept one or more collected hard example descriptors HEDi for use in the federated learning. A detailed example of accepting or rejecting hard example descriptors is given in FIG. 5. Upon acceptance, the coordinator system CS may provide an acceptance notice AN, 350, for the accepted hard example descriptor to the local system LS1 from which the hard example descriptor HEDi was collected.

Upon obtaining the acceptance notice AN from the coordinator system, the local system LS1 may be configured to use the hard example in the federated learning of the machine learnable model. For example, as illustrated in the figure, the federated learning based on the hard example may be performed in one or more iterations in which the local system LS1 may determining a model update MUj, 380, based on the hard example, and the coordinator system acting as a central server aggregates model updates MUj received from the respective local systems LSi and distributes an updated version MLM′, 370, of the machine learnable model to the local systems for future use. In this example, the federated learning system may be a centralized federated learning system; however, the provided techniques can be used in a decentralized federated learning system, or in a centralized federated learning system where the coordinator system CS is different from the central server, as well. The federated learning may comprise a verification by the coordinator system CS whether the updated machine learnable model MLM′ is sufficiently accurate, e.g., has improved accuracy or accuracy exceeding a threshold, as measured on the reference dataset RD. Provenance records may be kept for regulatory purposes.

Instead of or in addition to accepting hard examples for use in the federated learning itself, also one or more hard examples may be accepted for inclusion in a reference dataset RD, 330. The reference dataset RD may be maintained by the coordinator system CS and may be used to accept or reject future hard examples, and/or in the federated learning, etc. In such cases, the acceptance notice AN may indicate that the hard example is for inclusion in the reference dataset, and the respective local system LSi may upon receiving the acceptance notice provide the hard example to the coordinator system CS.

Accordingly, outlier data may be collected in a compliant way, e.g. under the appropriate legal basis, hard examples may be included in the reference dataset RD. Interestingly, using the provided techniques, only valuable/rare cases may need to be shared, performing much less data exchange while still ensuring a high-quality model and reference dataset.

A local system may use or more hard examples in the federated learning and may provide one or more further hard examples for inclusion in the reference dataset RD, but the same hard example is typically not used for both purposes.

The use of federated learning by federated learning system 300 may address relevant privacy concerns. Following the “model-to-data” paradigm of federated learning, model updates MUj may be exchanged as opposed to collecting all training data at a centralized location. Any appropriate federated learning framework can be used. Compared to traditional deployments of federated learning, the training may be stretched out in time, and there may be not be a coordinator who chooses a next local system for training. Instead, local systems themselves may determine hard examples and share them; these hard examples may then be used in the training only under certain conditions as determined by the coordinator system CS, e.g., only if enough similar hard examples are available.

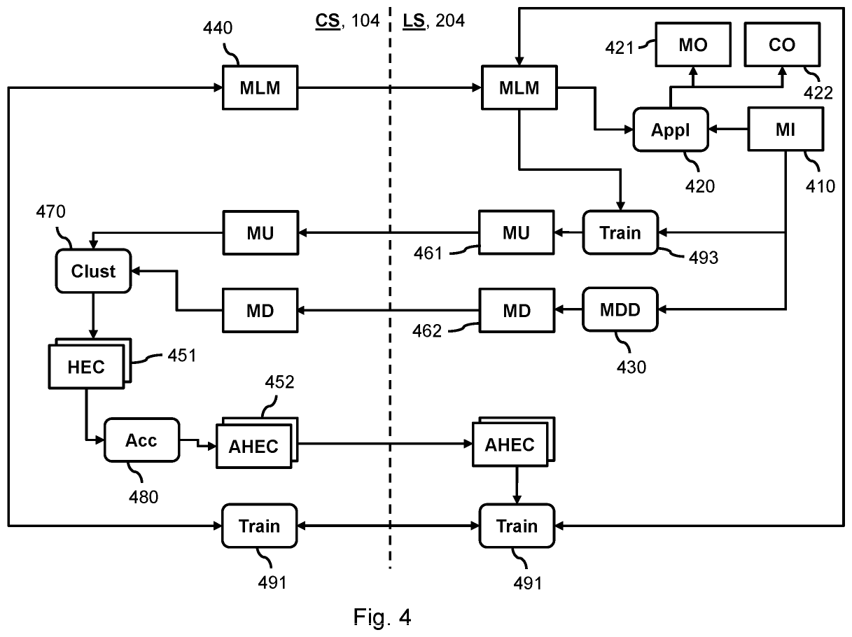

FIG. 4 shows a detailed, yet non-limiting, example of how to train a machine learnable model MLM, 440, by federated learning.

Shown in the figure is a local system LS, 204, e.g., based on local system 200 of FIG. 2 and/or a local system LSi of FIG. 3. Also shown is a coordinator system CS, 104, e.g., based on coordinator system 100 of FIG. 1 and/or coordinator system CS of FIG. 2. The local system LS may be configured to apply Appl, 420, the machine learnable model MLM, to model inputs MI, 410, resulting in a model input MO, 421. For example, the model input MI may be a medical image and the model output MO may be a classification of that medical image.

The local system LS may determine one or more model inputs MI, for example, model inputs to which the model is applied in regular operation of the machine learnable model MLM, to be hard examples. A hard example may be a model input, typically labelled and preferably manually labelled, that is determined to be potentially relevant for retraining the machine learnable model MLM, e.g., the model input may be determined to be a likely a rare case or model input that is erroneous or low quality.

For example, the model input MI may be determined to be a hard example based on a confidence score CO, 422, computed for the model input MI. The model input MI may be selected as a hard example if the confidence score is below a threshold, for example. This threshold may be higher than the classification threshold, e.g., the model MLM may output a classification MO while the model input MI is still considered to be a hard example. As illustrated in the figure, the confidence score CO may be determined by applying Appl the machine learnable model MLM. Many machine learnable models that are known per se may output a value that is indicative of the confidence of the model in its model output, e.g., a class probability or the like, and that can accordingly be used as the confidence score CO.

However, it is also possible for local system LS to determine the confidence score by using a separate anomaly detection model. The confidence score may be computed by the separate model according to a blind cross-check to avoid bias. For example, the confidence score may be based at least in part based on information not comprised in the hard example descriptor, e.g., additional metadata. The separate model may use information that is not comprised in the model input to make a more accurate assessment, e.g., metadata and/or historical records.

It is also possible to use other techniques instead of in addition to a confidence score to obtain hard examples. For example, one or more model inputs may be manually flagged as being hard examples, e.g., being of questionable quality. As an example, considering interpretation of CT scans as application of the system, a radiology department can choose CT images that are either judged to be of questionable quality, or that have a small confidence score. It is also possible that one or more model inputs are selected as hard examples by random selection, for example, to encourage variation in the set of hard examples, or because the labelling of such random cases may in any case happen as part of a regular review of the performance of the model. The hard examples are typically manually labelled, for example, as part of a periodic (e.g., monthly or quarterly) expert review, to ensure that they are correct.

Having selected model input MI as a hard example, the local system LS may be configured to determine metadata MD, 462, describing the hard example. In general, the metadata MD may comprise one or more features that are based on the model input MI and/or its label, and/or one or more features that are not based neither on the model input nor on its label.

The metadata MD may comprise a descriptor of a labeler of the hard example. This is useful because also the input provided by human annotators may contain errors, sometimes referred to as “type 1 errors”.

When applied in the clinical care practice, the metadata MD may further comprise information about health care professions associated with the model input MI, e.g., a care provider involved, a person making diagnosis corresponding to the model input, etc. For example, this information may include an experience level of the health care professional(s), or a degree of agreement about a labelling of the hard example, such as a STAPLE coefficient as disclosed in S. Warfield et al., “Simultaneous truth and performance level estimation (STAPLE): an algorithm for the validation of image segmentation”, DOI: 10.1109/TMI.2004.828354 (incorporated herein by reference). Such parameters are greatly beneficial to rank experts and reweight hard examples, in particular, to detect, flag, and reject “bad cases”. The metadata may further comprise information about a patient associated to the hard example, for example, about the patient being imaged, such as a patient diagnosis or a lab test result. Such information may be extracted from an EMR, for example.

The metadata MD may further comprise a timestamp of the model input MI, e.g., of when the medical image was captured or the record was entered, allowing to establish a similarity of hard examples based on this information. The metadata MD may further comprise a descriptor of the local system LS at which the hard example originated. All of this information may be useful to determine a cluster of similar hard examples and/or to determine whether hard examples are likely to be anomalous. The set of metadata MD to be determined may be determined in advance e.g., may be indicated in a metadata profile associated with the machine learnable model as disclosed in patent application PCT/EP2020/051647 titled “ASSOCIATING A POPULATION DESCRIPTOR WITH A TRAINED MODEL” (incorporated herein by reference insofar as the definition of the metadata profile is concerned), or as disclosed in patent application U.S. Ser. No. 16/547,880 titled “Determining features to be included in a risk assessment instrument” (incorporated herein by reference). The number of metadata features may be at most or at least, at most or at least 20, or at most or at least 50, for example.

The local system LS may further determine a model update MU, 461, for updating the model parameters of the machine learnable model MLM. For this, the normal procedure of the federated learning system for determining a model update MU may be used. During federated learning, such model updates are typically exchanged in multiple iterations to update the model; to determine the model update MU, the techniques used by the local system to determine the model update in one such iteration may be used. For example, the model update may comprise a gradient descent update to the model parameters. The model update MU may comprise an updated value or update for each trainable parameters of the machine learnable model, e.g., may comprise at least 1000, at least 10000, or at least 100000 updated value or updates. It is also possible to include only a subset of the updates or update values in the model update MU: this may save storage, bandwidth, and computation time, but may still provide sufficient information for the purpose of accepting or rejecting the hard example, in particular for the purpose of comparing model updates among each other.

The local system LS may provide a hard example descriptor comprising the model update MU and the metadata MD to the coordinator system CS. The hard example typically comprises a manual label, e.g., determined as part of a review process.

Accordingly, hard example descriptors may for example be periodically provided to the coordinator system CS, e.g., after a review of a batch of hard examples is completed, or one-by-one as new hard examples are determined. The local system LS may keep the hard examples at least until it has received notice of the coordinator system CS whether the hard examples are to be used for federated learning and/or providing as reference data.

The coordinator system CS may collect a set of hard example descriptors MU, MD from the local systems of the federated learning system. The system CS may determine whether or not to use the hard examples that are collected, and may thus be referred to as an oracle platform for accepting the hard examples. The collection system CS may perform the procedure for determining which hard example descriptors to accept, upon receiving a new hard example descriptor, upon receiving a predefined number of new hard example descriptors, or periodically, for example. As also discussed with respect to FIG. 5, the procedure may lead to one or more hard example descriptors being accepted that were previously already processed and rejected, for example, because at that point no similar additional hard examples were available. Accordingly, the coordinator system CS typically keeps hard example descriptors that are not at that point accepted, for later use.

Interestingly, the determination which hard example descriptors to accept or reject may be made based on the hard example descriptors without access to the hard examples, e.g., the model inputs and labels, themselves. The system may aim to collect fascinoma and filter out low-quality data, and thereby prevent deterioration of the models' performance on the curated reference dataset.

From the set of collected hard example descriptors, the coordinator system LS may select a cluster HEC, 451 of similar hard example descriptors. The cluster may comprise multiple hard example descriptors. The clustering is typically based on the metadata MD and model update MU of a hard example descriptor. For example, the cluster may be obtained according to a clustering algorithm that divides the set of hard example descriptors into clusters, but it is also possible to select the cluster without clustering the remaining hard example descriptors, e.g., by selecting a hard example descriptor and including hard example descriptors that are within a given distance from the hard example according to a distance measure. Detailed examples are discussed with respect to FIG. 5.

Given the selected cluster HEC, the coordinator system CS may determine Acc, 480 whether or not to accept the cluster, based on the model updates MU and metadata MD of the hard example descriptors of the cluster. Detailed examples of this are discussed with respect to FIG. 5. If the cluster is accepted, the hard examples of the accepted cluster AHEC, 452, may be used in the federated learning and/or included in a reference dataset kept by the coordinator system CS (not shown in this figure).

In particular, as shown in the figure, one or more hard examples of the accepted cluster AHEC may be used in a training operation Train, 491, of the federated learning system. The training operation Train may for example be initiated by the coordinator system CS when a cluster AHEC has been accepted, or may be performed periodically using all clusters AHEC accepted during the past period, etc. The training Train may be carried out as known per se for federated learning. At least the local system LS, having access to the model input MI and also the labelling typically used in the federated learning, may participate in the training operation. As shown in the figure, depending on the federated learning techniques used, also the coordination system CS may participate, for example as central system. Also other local systems may contribute respective hard examples and participate in the training operation Train. As is known, the training typically uses additional training data, e.g., previously accepted hard examples, in addition to the newly accepted hard examples.

While not shown in the figure, one or more further hard examples of the accepted cluster AHEC may be selected by the coordinator system CS for inclusion in a reference dataset, as also discussed with respect to FIG. 3, for example. In such cases, the coordinator system CS may provide an acceptance notice to the local system LS which may then provide the model input MI and/or its label to the coordinator system CS.

FIG. 5 shows a detailed, yet non-limiting, example of how to accept a set of hard example descriptors for use in federated learning and/or for inclusion in a reference dataset. For example, the techniques of this figure may be used to implement the clustering operation Clust, 470 and the acceptance operation Acc, 480, of FIG. 4.

The figure shows a set of collected hard example descriptors 560. A hard example descriptor may comprise metadata MD about a hard example, as well as a model update MU for updating model parameters of a machine learnable model from the hard example. The obtained metadata MD and the models' updates MU may be used to find similar hard examples distributed across the local systems.

In particular, in this example, a cluster of similar hard example descriptors may be determined based on determining Dist, 571, a pairwise distance between hard example descriptors. In particular, as illustrated in the figure, an affinity matrix 572, also known as a similarity matrix, of pairwise distances may be determined. To determine a pairwise distance, the metadata MD and the updates MU may be combined to form a feature vector representing the hard example, and a similarity S between these feature vectors may be computed, e.g., as negative distance between the vectors S=1−D. For example, the distance can be a Ln-distance for an appropriate value of n, a Levenshtein distance, a Hamming distance, etc. The distance calculation Dist can also compute different measures for different groups of the parameters and aggregate them aggregates it. The computed distances can for example be expressed as values between 0 and 1 with 1 indicating minimal distance and maximal correspondence.

Based on the pairwise distances 572, a distance-based clustering CI, 573, may be performed to determine one or more clusters from the set of hard example descriptors 560. As an illustration, shown are a cluster 574 of five hard example descriptors; a cluster 575 of two hard example descriptors; and a cluster 576 of just one hard example descriptor. For example, the distance-based clustering can be spectral clustering, density-based spatial clustering of applications with noise (DBSCAN) clustering, ordering points to identify the clustering structure (OPTICS) clustering, etc. It is also possible to use a non-distance-based clustering. It is also not necessary to complete the clustering, e.g., if a large enough cluster is found then the clustering of the remaining clusters can be stopped, with the large enough cluster possibly still being extended with similar examples.

Given a cluster 574, it may be determined CA?, 580, whether to accept or reject the cluster. This process may be repeated for each determined cluster. A number of factors may be taken into account in this determination. As illustrated in the figure, the determination may be implemented as a decision tree.

A first factor of the determination may be a check CPs?, 581, of one or more cluster parameters of the cluster. Specifically, it may be checked whether the size of the cluster and/or the number of local systems contributing to the cluster is sufficient. Both a large enough cluster and a large enough number of contributing systems may indicate a low chance of having an artifact. The larger the number of contributing systems, the smaller the cluster size that may be tolerated. For example, the check may pass, Y, if the number of local systems is at least a given threshold X (e.g., X=5), and/or if the size of the cluster is at least a given threshold Y (e.g., Y=20). Otherwise, the check may fail, N, in which the cluster may directly be rejected Rej, 593.

Generally, the minimal size of the cluster depends on the model size and nature of the data. A cluster size may be configured such that a sufficient number of examples for model retraining is available and overfitting is prevented, e.g., the minimal cluster size may be at least 10⁻⁴ times the size of the training set Dₜᵣₐᵢₙ, or at least 5 or 10. Another advantage of checking cluster parameters is that filtering small clusters may help to prevent unexpected model changes that might cause deterioration of the accuracy.

A further factor of the determination may be a check Eff?, 582, of an estimated effect of the cluster on the accuracy of the machine learnable model. The effect may indicate a risk of the model performance deteriorating after using the cluster for training. This effect being low L may lead to one or more, preferably all, of the hard examples of the cluster being used for the training Train, 591.

To determine the estimated effect, techniques may be used that are known per se for estimating the influence of a training example on a prediction made by the model, e.g., computed implicitly from the model updates using derived updates. For example, tracing influence may be used, as known from G. Pruthi et al., “Estimating Training Data Influence by Tracing Gradient Descent” (available at https://arxiv.org/abs/2002.08484 and incorporated herein by reference), or an influence function may be computed, as known from P. W. Koh et al., “ Understanding Black-box Predictions via Influence Functions” (available at https://arxiv.org/abs/1703.04730 and incorporated herein by reference). Using such techniques and based on the reference dataset maintained by the coordinator system, a difference in model accuracy before and after the update, measured per each testing example, may be estimated. Model updates which may deteriorate the performance of the model on a set of testing cases may be held off and tagged as “suspicious”.

Another factor of the determination may be a check Repr?, 583, of the degree to which the cluster is representative of the reference dataset. The degree of representativeness may indicate whether the cluster is likely to be a rare case. This check is preferably performed on the metadata of the hard examples only. Interestingly, the cluster may be accepted based on the cluster being sufficiently unrepresentative of the reference dataset, e.g., based on the representativeness being below a given threshold. If the cluster is sufficiently unrepresentative, N, one or more of the hard examples of the clusters may be used for the training Train, and preferably, one or more further hard examples of the cluster may be included Val, 592, in the validation dataset. If the cluster is not sufficiently unrepresentative, e.g., if its representativeness exceeds a threshold, it may be rejected, Rej.

If the cluster is sufficiently unrepresentative, it may be an outlier, and may thus be beneficial for the model even if it leads to models' accuracy deterioration on the main curated dataset, which may not contain a representative case yet. By using the cluster both for training and for inclusion in the reference dataset, both the model and its reference dataset may be improved.

The check Repr? may be performed by using technique known per se to determine whether the hard examples of the cluster are in-distribution of the reference dataset. For example, a generative model may be trained of the reference dataset, with the checking being performed by computing probabilities of the hard examples being generated according to the generative model. The check can also be based on per-feature probability distributions for the respective features of the metadata, for example. Before performing the check Repr?, expected bounds may be derived for metaparameters from the reference dataset. When performing the check, it may be checked if the hard examples lie withing the bounds (common case, is rejected Rej) or represent an outlier (interesting case, is used for Train and/or validation Val.

The check Repr? may further comprise checking that the cluster is not anomalous, in other words, if it is sufficiently plausible, e.g., its anomaly score does is below a threshold. If the cluster is too unrepresentative of the reference dataset, it may be rejected Rej as being a likely anomaly. For example, the check Repr? may check whether the representativeness of the cluster to the reference dataset lies in a particular interval. This further decreases the probability of low-quality data being accepted.

While shown as three separate decisions CPs?, Eff?, Repr? of a fixed decision tree, it is also possible to combine the respective acceptance/rejection fashions in a different way, e.g., by applying a machine learnable model that outputs the decision, such as a trained decision tree, a support vector machine, etc. It is also possible, e.g., in addition to the described decision procedure, to take into account one or more custom rules when deciding whether to accept or reject a cluster. For example, the analysis may be configured to accept or reject a cluster based on the type of medical personnel and/or institution related to a hard example of the cluster, e.g. a hard example involving a top expert or a university medical center is more likely to be a rare diagnosis or procedure not seen before, while a case annotated by a student or at a local hospital is more likely to have quality issues.

Interestingly, based on the analysis of the hard example descriptors as described herein, the coordinator system may also detect a domain shift (also known as an input shift). For example, it may be detected that the number of hard example descriptors from a particular local system that are rejected, has increased. As an example, the system may face a consistent new (sub)cohort previously not seen by the ML model (e.g. Asian-descent patient cohort appearing on a site that is within a Chinatown district, while the original model was only trained on Caucasian population). In such cases, an alert may be raised to a user of the coordinator system to take appropriate action, e.g., to manually flag these hard examples for use in the training Train and/or validation Val, or to collect more similar data.

Generally, the analysis steps Dist, CI, CA?, may be repeated, e.g., every time a new batch of hard example descriptors is received, or periodically, etc. If the coordinator system accepts a cluster 574, e.g., it passes tests CPs?, Eff?, and/or Repr? according to the decision procedure shown in the figure, federated re-training Train may be initiated. The coordinator system may trigger the model training procedure on the local systems that have contributed hard examples to the accepted clusters. The local systems may for example provide updates that may be aggregated by the coordinator system, e.g., in one or more iterations.

The way the analysis Dist, CI, CA? is carried out, may be further refined based on feedback on the decision of whether or not a cluster was accepted. For example, decisions to accept or reject a cluster may be reviewed by a human or may be validated by later obtained data, such as later additions to the reference dataset. For example, regular expert validation may be carried out, in which the accept/reject decisions may be assessed by human experts, e.g., when the confidence is lower that a threshold or on a regular basis to ensure quality control. For example, the feedback may be based on events in the future (e.g., for time series), ground truth that becomes known only later (e.g. for pathology results), a discrepancy between the inference result and a radiologist report that is automatically detected, etc. The feedback may be used to automatically adjust criteria (in other words, parameters) of the analysis, such as the minimal cluster size, the minimum number of local systems, and/or the minimal or maximal unrepresentativeness used, or parameters of a machine learnable model used to make the decision.

As also discussed elsewhere, based on the analysis, hard examples(s) of an accepted cluster may be used for training Train by federated learning or for inclusion Val in a reference dataset. Hard examples that are not in an accepted cluster, e.g., are rejected Rej or not included in an analyzed cluster at all, may be kept and used in later analysis. This may be helpful to extend the training and testing datasets in the future, for example, a hard example of a cluster 576 that is considered too small by check CP? may in a later clustering CI be combined with other, e.g., newly obtained, hard example descriptors in a new cluster that may be large enough and may then be accepted.

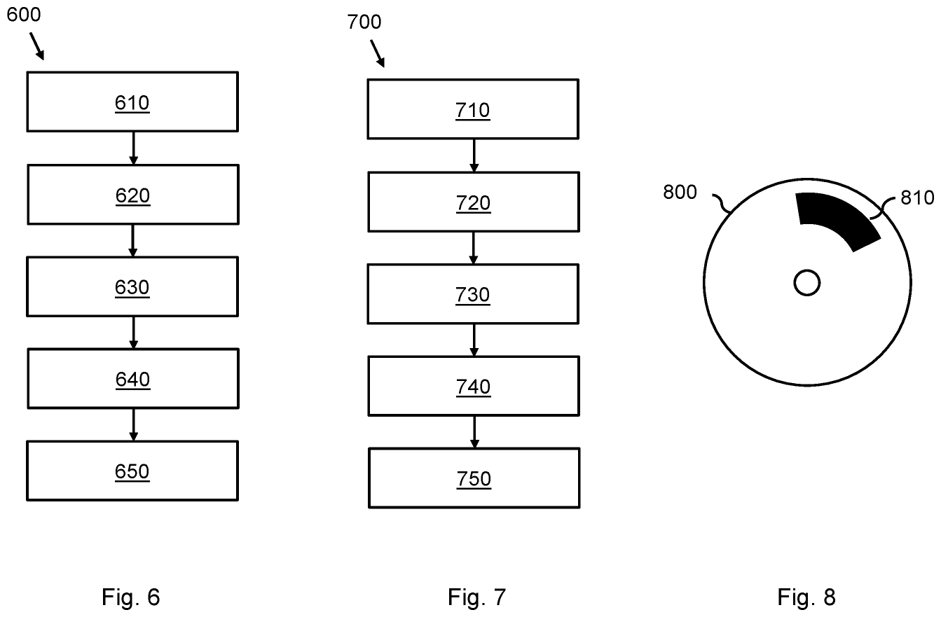

FIG. 6 shows a block-diagram of computer-implemented method 600 of operating a local system of a federated learning system. The federated learning system is for federated learning of a machine learnable model. The federated learning system may comprise a coordinator system. The method 600 may correspond to an operation of the system 200 of FIG. 2, the system 201 of FIG. 3, or the system 204 of FIG. 4. However, this is not a limitation, in that the method 600 may also be performed using another system, apparatus or device.

The method 600 may comprise, in an operation titled “ACCESS MODEL”, accessing 610 model data representing model parameters of the machine learnable model. The method 600 may comprise, in an operation titled “OBTAIN HARD EXAMPLE”, obtaining 620 a hard example for the machine learnable model. The method 600 may comprise, in an operation titled “DETERMINE MODEL UPDATE”, determining 630 a model update for updating the model parameters of the machine learnable model from the hard example. The method 600 may comprise, in an operation titled “PROVIDE UPDATE +METADATA”, providing 640, to the coordinator system, a hard example descriptor comprising the model update and metadata describing the hard example. The method 600 may comprise, in an operation titled “USE HARD EXAMPLE”, upon obtaining, from the coordinator system, an acceptance notice for the hard example descriptor, using 650 the hard example in the federated learning of the machine learnable model.

FIG. 7 shows a block-diagram of computer-implemented method 700 of operating a coordinator system of a federated learning system. The federated learning system may be for federated learning of a machine learnable model. The federated learning system may comprise multiple local systems. The method 700 may correspond to an operation of the system 100 of FIG. 1, the system 101 of FIG. 3, or the system 104 of FIG. 4. However, this is not a limitation, in that the method 800 may also be performed using another system, apparatus or device.

The method 700 may comprise, in an operation titled “ACCESS HARD EXAMPLE DESCRIPTORS”, accessing 710 a set of hard example descriptors. The method 700 may comprise, in an operation titled “COLLECT”, collecting 720 the set of hard example descriptors from the multiple local systems. The method 700 may comprise, in an operation titled “CLUSTER”, selecting 730 a cluster of similar hard example descriptors from the set of hard example descriptors. The method 700 may comprise, in an operation titled “ACCEPT”, based on model updates and metadata comprised in the hard example descriptors of the cluster, accepting 740 the cluster for use in the federated learning. The method 700 may comprise, in an operation titled “PROVIDE ACCEPTANCE NOTICE”, providing 750 an acceptance notice for a hard example descriptor of the cluster to the local system from which the hard example descriptor was collected.

It will be appreciated that, in general, the operations of method 600 of FIG. 6, and method 700 of FIG. 7 may be performed in any suitable order, e.g., consecutively, simultaneously, or a combination thereof, subject to, where applicable, a particular order being necessitated, e.g., by input/output relations. Some or all of the methods may also be combined, e.g., method 600 of operating a local system and method 700 of operating a coordinator system may be implemented to run simultaneously at a single system.

The method(s) may be implemented on a computer as a computer implemented method, as dedicated hardware, or as a combination of both. As also illustrated in FIG. 8, instructions for the computer, e.g., executable code, may be stored on a computer readable medium 800, e.g., in the form of a series 810 of machine-readable physical marks and/or as a series of elements having different electrical, e.g., magnetic, or optical properties or values. The medium 800 may be transitory or non-transitory. Examples of computer readable mediums include memory devices, optical storage devices, integrated circuits, servers, online software, etc. FIG. 8 shows an optical disc 800. Alternatively, the computer readable medium 800 may comprise model data 810 representing model parameters of a machine learnable model trained by a federated learning system as described herein.

Examples, embodiments or optional features, whether indicated as non-limiting or not, are not to be understood as limiting the invention as claimed.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or stages other than those stated in a claim. The article “a” or “an” preceding an element does not exclude the presence of a plurality of such elements. Expressions such as “at least one of” when preceding a list or group of elements represent a selection of all or of any subset of elements from the list or group. For example, the expression, “at least one of A, B, and C” should be understood as including only A, only B, only C, both A and B, both A and C, both B and C, or all of A, B, and C. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A federated learning system for federated learning of a machine learnable model, wherein the federated learning system comprises multiple local systems and a coordinator system,
wherein a local system of the multiple local systems is configured to:
obtain a hard example for the machine learnable model;
determine a model update for updating model parameters of the machine learnable model from the hard example;
provide, to the coordinator system, a hard example descriptor comprising the model update and metadata describing the hard example; and
upon obtaining, from the coordinator system, an acceptance notice for the hard example descriptor, use the hard example in the federated learning of the machine learnable model;
wherein the coordinator system is configured to:
collect a set of hard example descriptors from the multiple local systems;
select a cluster of similar hard example descriptors from the set of hard example descriptors;
based on the model updates and the metadata comprised in the hard example descriptors of the cluster, accept the cluster for use in the federated learning; and
provide an acceptance notice for a hard example descriptor of the cluster to the local system from which the hard example descriptor was collected.

2. The system of claim 1, wherein the local system is further configured to apply the machine learnable model to a model input comprising healthcare data about a patient, for example, to a medical image.

3. The system of claim 1, wherein the coordinator system is configured to accept the cluster based on one or more of a size of the cluster, a number of local systems contributing to the cluster, an estimated effect of the cluster on an accuracy of the machine learnable model and a degree to which the cluster is representative of a reference dataset.

4. The system of claim 3, wherein the coordinator system is configured to accept the cluster based on the cluster being sufficiently unrepresentative of the reference dataset.

5. The system of claim 1, wherein the acceptance notice provided to the local system is for use of the hard example in the federated learning, and wherein the coordinator system is configured to provide a further acceptance notice for a further hard example descriptor of a further hard example of the cluster for inclusion of the further hard example in a reference dataset.

6. The system of claim 1, wherein the local system is configured to obtain a model input for the machine learnable model; compute a confidence score for the model input; and select the model input as the hard example based on the confidence score being below a threshold.

7. The system of claim 1, wherein the metadata comprises data that is derived from neither a model input nor a label of the hard example.

8. The system of claim 1, wherein the metadata comprises one or more of a timestamp, a descriptor of the local system, a descriptor of a labeler of the hard example, a degree of agreement about a labelling of the hard example, information about a health care professional associated with the hard example, information about a patient associated to the hard example, and information about image acquisition settings the hard example.

9. The system of claim 1, wherein the coordinator system is configured to obtain feedback on the accepted cluster and to update criteria for accepting clusters based on the feedback.

10. The system of claim 1, wherein the coordinator system is configured to keep a hard example descriptor not comprised in a selected cluster or comprised in a non-accepted cluster, and use the kept hard example descriptor in a further cluster selection and acceptance.

11. A local system for use in the federated learning system of claim 1, wherein the federated learning system is for federated learning of a machine learnable model, wherein the federated learning system comprises a coordinator system, wherein the local system comprises:
a data interface for accessing model data representing model parameters of the machine learnable model;
a processor subsystem configured to:
obtain a hard example for the machine learnable model;
determine a model update for updating the model parameters of the machine learnable model from the hard example;
provide, to the coordinator system, a hard example descriptor comprising the model update and metadata describing the hard example; and
upon obtaining, from the coordinator system, an acceptance notice for the hard example descriptor, use the hard example in the federated learning of the machine learnable model.

12. A coordinator system for use in the federated learning system of claim 1, wherein the federated learning system is for federated learning of a machine learnable model, wherein the federated learning system comprises multiple local systems, wherein the coordinator system comprises:
a data interface for accessing a set of hard example descriptors;
a processor subsystem configured to:
collect the set of hard example descriptors from the multiple local systems;
select a cluster of similar hard example descriptors from the set of hard example descriptors;
based on model updates and metadata comprised in the hard example descriptors of the cluster, accept the cluster for use in the federated learning; and
provide an acceptance notice for a hard example descriptor of the cluster to the local system from which the hard example descriptor was collected.

13. A computer-implemented method of operating a local system of the federated learning system of claim 1, wherein the federated learning system is for federated learning of a machine learnable model, wherein the federated learning system comprises a coordinator system, wherein the method comprises:
accessing model data representing model parameters of the machine learnable model;
obtaining a hard example for the machine learnable model;
determining a model update for updating the model parameters of the machine learnable model from the hard example;
providing to the coordinator system, a hard example descriptor comprising the model update and metadata describing the hard example; and
upon obtaining, from the coordinator system, an acceptance notice for the hard example descriptor, using the hard example in the federated learning of the machine learnable model.

14. A computer-implemented method of operating a coordinator system of the federated learning system of claim 1, wherein the federated learning system is for federated learning of a machine learnable model, wherein the federated learning system comprises multiple local systems, wherein the method comprises:
accessing a set of hard example descriptors;
collecting the set of hard example descriptors from the multiple local systems;
selecting a cluster of similar hard example descriptors from the set of hard example descriptors;
based on model updates and metadata comprised in the hard example descriptors of the cluster, accepting the cluster for use in the federated learning; and
providing an acceptance notice for a hard example descriptor of the cluster to the local system from which the hard example descriptor was collected.

15. A transitory or non-transitory computer-readable medium comprising data representing
instructions which, when executed by a processor system, cause the processor system to perform the computer-implemented method according to and/or
model data representing model parameters of a machine learnable model trained by the federated learning system of claim 1.
